(19) United States
(12) Patent Application Publication
(10) Pub. No.: US 2026/0278895 A1
(43) Pub. Date: Sep. 17, 2026

(54) METHODS, SYSTEMS, AND DEVICES FOR METAL ARTIFACT REDUCTION

(71) Applicant: Siemens Healthineers AG, Forchheim (DE)

(72) Inventors: Julian ANHAUS, Fuerth (DE); Alexander BROST, Erlangen (DE); Christian HOFMANN, Erlangen (DE)

(73) Assignee: Siemens Healthineers AG, Forchheim (DE)

(21) Appl. No.: 19/564,347

(22) Filed: Mar. 12, 2026

(30) Foreign Application Priority Data
Mar. 12, 2025 (DE) 10 2025 109 387.3

(51) Int. Cl. G06T 12/30; G06F 3/04845; G06T 5/50; G06T 7/12; G06T 15/08; G16H 15/00; G16H 30/20; G16H 30/40

(52) U.S. Cl. CPC G06T 12/30; G06F 3/04845; G06T 5/50; G06T 7/12; G06T 15/08; G16H 15/00; G16H 30/20; G16H 30/40; G06T 2207/20221

(57) ABSTRACT

One or more example embodiments relates to a computer-implemented technique for providing a metal artifact reduced volumetric image reconstruction. The technique may be implemented in a PACS, DICOM node, or any other computing system outside a scanner. A method comprises receiving at least one volumetric image reconstruction of a medical scan from the scanner pertaining to a volumetric medical imaging modality. Via a metal detection algorithm, a metal comprised within the at least one volumetric image reconstruction is detected. Responsive to the detecting of the metal and via a (e.g., deep learning) metal artifact reduction, MAR, algorithm, MAR on the received at least one volumetric image reconstruction is performed. The metal artifact reduced at least one volumetric image reconstruction is provided, for example, to a PACS or for viewing.

### CROSS-REFERENCE TO RELATED APPLICATION(S)

The present application claims priority under 35 U.S.C. § 119 to German Patent Application No. 10 2025 109 387.3, filed Mar. 12, 2025, the entire contents of which is incorporated herein by reference.

### FIELD

One or more example embodiments relates to a technique for providing a metal artifact reduced volumetric image reconstruction, in particular comprising a method, a computing device, a system comprising the computing device, and a computer program product.

### RELATED ART

Metal implants are common in computed tomography (CT) scans. Every year in Germany, a total of 47 million dental fillings are inserted, in addition to almost 440,000 implantations of endoprostheses in the extremities and hips [1,2]. Metal implants are a problem in CT imaging, as they introduce severe imaging artifacts (beam hardening, photon starvation, and scattering) to the reconstructed image [3]. Various metal artifact reduction (MAR) algorithms, which reduce related artifacts, are currently deployed and performed on CT systems by different manufacturers. There, the MAR is applied to the uncorrected volume. Both, the uncorrected and MAR-corrected series, are then reconstructed and stored, currently only uniquely identified by the series description. This introduces workflow-related problems to the overall imaging chain, as the metal artifact-corrected series need to be reconstructed, manually re-named within the series description, sent to the PACS, and then re-identified and manually selected during viewing. Additional scanner-deployed reconstruction tasks (often protocol-induced) further increase the number of permutations of non MAR-corrected and MAR-corrected image series, such as providing multiple reconstruction kernels, orientations, or spectral postprocessing (SPP) images, including virtual monoenergetic images (VMI). Currently, each of those series is sent at least twice (non MAR-corrected and MAR-corrected), effectively doubling the number of series sent from the modality to the PACS. Those steps restrict an automatic clinical workflow and require a large amount of bandwidth between the scanner and the PACS. Conventional iterative MARs, such as Siemens iterative MAR (iMAR) [4], further intensify this problem: Multiple factors have an influence on the expression and severity of the artifact strength in the reconstructed image, such as the implant's material, size, shape, or tissue material composition around the implant. For this reason, iMAR provides eight user-selectable implant-specific presets, further increasing the multiplication factor of the reconstructed, sent, stored, and viewed image series.

Without additional metal artifact reduction, it is unlikely that the CT image can still be used for diagnostic purpose. MAR algorithms can be used to reduce respective artifacts in the reconstructed image. Among various different approaches, the sinogram interpolation in a normalized sinogram domain has widely been established by CT vendors (such as NMAR: normalized MAR [5]). Sinogram interpolation describes the modification of the 2D parallel-beam sinogram by modifying values of the metal trace, i.e., all projections which include metal. The image quality after interpolation is strongly dependent on the computed prior image, which is used for the sinogram normalization. The prior image should include an optimal segmentation of the bone, air, and soft tissue in the CT image. The effect of an incomplete prior image has partially been described in [7]. Currently, simple Hounsfield (HU) thresholds are applied to the uncorrected image to detect bone, air, and soft tissue for the prior image, which is an unstable method in the presence of metal artifacts. The presence of contrast agent in the CT image (with similar HU values as the bone) further enhances this problem.

Conventionally, the following steps are performed in the presence of metal artifacts on a CT system, as shown in FIG. 3. The operator of a medical scanner (also: modality) checks if metal is present within the CT scan range and then manually enables additional MAR, usually as a binary manual input. For Siemens CT scanners, in addition, the operator specifies the implant type by manual input on a UI, selecting between eight (8) predefined implant-specific presets: dental fillings, neurocoil, shoulder implant, pacemaker, spine implant, thoracic coil, hip implant, and/or extremity implant. The operator can manually adapt the series description, based on the selected preset. Otherwise, the MAR-corrected series can't be re-identified anymore afterward. Additional reconstruction tasks are usually also duplicated, sent, and stored with the number of required MAR settings, at least doubling the number of reconstructions. Each series description of those additional series should be manually renamed for later reidentification. This includes multi-plane reformats (MPRs, such as axial, sagittal, or coronal reformats or different slice thickness reconstructions) and SPP reconstructions, such as VMIs, or virtual non-contrast images. On a photon-counting CT system, like the NAEOTOM Alpha, manufactured by the Applicant, each threshold image is computed separately by iMAR [6]. Additional reconstructions with different kernels are performed. During the diagnostic reading process at the PACS, both the uncorrected and the MAR-corrected series are opened manually. Each additional available MAR series (from the previous point) adds complexity to the manual reading process.

Conventionally, manufacturers, such as Siemens, apply an iterative loop in its cleared product (e.g., iMAR) to maximize the amount of bone and air present in the prior image, as shown FIG. 5. The iterative loop is used to apply converging HU thresholds in each iteration, based on the assumption of reduced artifacts within each iteration. This approach introduces various problems to the algorithm design and consequently to the image quality after MAR. In the presence of metal artifacts, applying HU thresholds to segment bone is unstable and either leads to the loss of detail after correction (if not enough bone is segmented) or the introduction of new artifacts after correction (if metal artifacts are misclassified as bone). Additionally, the presence of contrast agent is not incorporated by applying HU thresholds, so that parts of the contrast-flooded organs are also incorporated in the bone mask of the prior image. This effect is depending on the type of contrast agent, the contrast phase, and the contrast agent saturation. Further additionally, the HU thresholds for the tissue types (air and bone) of the prior image must be defined manually. As bone types and thus the bone's HU values differ for different body regions, Siemens' iMAR introduces user-selectable strength settings to adapt those parameters, which introduce workflow-related problems. Most of the parameters incorporated in the user-selectable strength settings are used to control the iterative correction loop and the metal detection.

### SUMMARY

One or more example embodiments provides a solution for improving an image quality after metal artifact reduction of volumetric medical imaging, including for imaging using a contrast agent. Alternatively or in addition, one or more example embodiments reduces a computational load on a medical scanner, to reduce data traffic originating from the medical scanner, and/or to reduce an amount of computing resources for volumetric image reconstructions. Alternatively or in addition, one or more example embodiments automatizes a clinical workflow related to volumetric medica imaging and/or to reduce a necessary number of manual steps. Alternatively or in addition, one or more example embodiments provides better comparability when comparing scans with metal artifacts in-between different scanner manufacturers and/or vendors.

### BRIEF DESCRIPTION OF THE DRAWINGS

Properties, features and advantages of example embodiments, as well as the manner they are achieved, become clearer and more understandable in the light of the following description, which will be described in more detail in the context of the drawings.

FIG. 1 is a flow chart of a method for providing a metal artifact reduced (MAR) volumetric image reconstruction according to a preferred embodiment of the present invention;

FIG. 2 is an overview of the structure and architecture of a computing device for providing a MAR volumetric image reconstruction according to a preferred embodiment of the present invention, which computing device may be configured for performing the method of FIG. 1;

FIG. 3 shows an exemplary clinical workflow between the modality (CT) and the PACS according to the prior art;

FIG. 4 shows an exemplary embodiment of the new imaging chain for the PACS- or cloud-deployed MAR framework, combined with a DL-based metal artifact detection system, in particular according to the general method of FIG. 1;

FIG. 5 shows an exemplary conventionally used framework within Siemens' iMAR to optimize the segmented tissue in the prior image, which is used for the normalization of the sinogram domain before interpolating linearly, e.g., according to the description in [4]. All steps within the prior image calculation and the metal detection according to the prior art are purely based on HU-thresholds;

FIG. 6 schematically illustrates a further embodiment of the method of FIG. 1, which dispenses with a need to manually set Hounsfield unit (HU) thresholds for performing metal artifact reduction;

FIGS. 7A, 7B, 7C and 7D exemplarily shows a comparison between different window settings and HU thresholds;

FIG. 8 schematically illustrates that an image quality after metal artifact reduction strongly depends on the quality of the uncorrected image; and

FIGS. 9A, 9B and 9C exemplarily illustrate the influence of the segmented prior image to the final image quality after metal artifact reduction.

### DETAILED DESCRIPTION

In the following, one or more example embodiments will be described with respect to the claimed method. Features, advantages or alternative embodiments, mentioned with respect to the method, can be assigned to the other claimed objects (e.g., a device, in particular the computing device, the system, the computer program, or a computer program product), and vice versa. In other words, the device (and/or the system) can be improved with features described or claimed in the context of the method, and vice versa. In this case, the functional features of the method are embodied by structural units of the device and/or system and vice versa, respectively. The method may refer to a software implementation and the device (and/or system) may refer to a hardware implementation (e.g., with a spatial physical structure) or a virtualization thereof. Generally, in computer science a software implementation and a corresponding hardware implementation (e.g., as an embedded system) are equivalent. Thus, for example, a method step for “storing” data may be performed with a storage unit and respective instructions to write data into the storage. For the sake of avoiding redundancy, although the device (and/or system) may also be used in the alternative embodiments described with reference to the method, these embodiments are not explicitly described again for the device (and/or system). In principle, the respective device or apparatus claim (and/or system claim) is configured to carry out the claimed method.

As to a method aspect, a (in particular computer-implemented) method for providing a metal artifact reduced volumetric image reconstruction is provided. The method may in particular be performed in a picture archiving and communication system (PACS) of a medical facility. The method comprises a step of receiving at least one volumetric image reconstruction of a medical scan from a scanner pertaining to a volumetric medical imaging modality. The method further comprises a step of detecting a metal comprised within the at least one volumetric image reconstruction. The detecting is performed via a (e.g., deep learning) metal detection algorithm. The method further comprises a step of performing a metal artifact reduction on the received at least one volumetric image reconstruction. The metal artifact reduction (MAR) is performed responsive to the detecting of the metal. The metal artifact reduction is performed via a, in particular deep learning (DL), MAR algorithm. The method still further comprises a step of providing the metal artifact reduced at least one volumetric image reconstruction.

By the inventive technique, performing metal artifact reduced (MAR) volumetric image reconstructions (also: MAR reconstructions and/or corrected reconstructions) may be moved from the scanner (also: medical scanner, and/or modality) to (or in direction towards) a PACS of a medical facility (e.g., a hospital, a radiology department, and/or a medical practice), in which the scanner is located. Alternatively or in addition, performing the MAR reconstructions may be moved to a cloud server or server network (e.g., server(s) owned by the medical facility), and/or to a digital imaging and communications in medicine (DICOM) node. Thereby, computing resources (in particular processing and/or memory resources) at the scanner can be reduced, and/or a computational load at the scanner can be reduced. Alternatively or in addition, a network load (and/or an amount of data to be transmitted from the scanner, in particular to the PACS and/or any digital storage) can be reduced. In case MAR reconstructions are only performed upon demand (e.g., subject to a request by a user and/or the user's workflow), moreover, computing resources, such as processing resources and/or memory resources, can be saved in a computing cloud (and/or on the cloud server), a DICOM node, and/or the PACS. Alternatively or in addition, MAR reconstructions by the inventive technique may be performed on pre-existing uncorrected volumetric image reconstructions, which have been acquired and stored before the implementation of the inventive technique. Thereby, in particular a workflow for comparing prior studies with newly acquired volumetric image reconstructions can be improved. Further alternatively or in addition, performing MAR reconstructions (in particular solely) based on the volumetric image reconstruction and/or in the PACS (and/or at a DICOM node, and/or in a cloud or server owned by the medical facility) is advantageously manufacturer (and/or vendor) agnostic, e.g., independent of any specific software used for pre-processing (and/or processing) raw data obtained from the scanner.

In some embodiments, performing the MAR volumetric image reconstruction (briefly: MAR reconstruction) may be as required (e.g., depending on a user's, such as a radiologist's or oncologist's, workflow), and/or the MAR reconstructions need not be permanently saved. Thereby, digital storage space can be saved. This scenario may in particular apply to prior studies stored in the PACS.

The inventive technique may comprise a postprocessing framework for metal artifact reduction, and/or may make use of a retrospective (in particular DL-based) metal artifact reduction.

By moving the performing of the metal artifact reduction away from the scanner, and/or by using the (e.g., DL) metal detection algorithm and subsequently the (e.g., DL) MAR algorithm to obtain the MAR reconstruction, a clinical workflow can be significantly simplified. In particular, user input (in particular from an operator of the scanner) at the scanner regarding the metal can be dispensed with.

Alternatively or in addition, a workflow can be simplified at a viewing station and/or for a user, in particular a medical practitioner, such as a radiologist or oncologist. In particular, the MAR reconstruction according to the inventive technique may be automatically performed and opened at the viewing station (and/or retrieved from its digital storage, such as from the PACS), reducing a need for the user to manually search for the corresponding stored file.

The viewing station may comprise a user interface (UI), in particular a graphical user interface (GUI).

The viewing station may be used by a medical expert, such as a radiologist or oncologist, e.g., in an office space and/or any location, which is in particular different from the location of the scanner.

By moving the MAR reconstruction away from the scanner, the inventive technique can be performed for medical scanners from different manufacturers and/or from different vendors. Alternatively or in addition, the inventive technique renders the MAR reconstruction independent of the local reconstruction software at the scanner.

By using the (e.g., DL) metal detection algorithm in combination with the (in particular DL-based) metal artifact reduction, an image quality of the MAR reconstruction can be enhanced, facilitating an improved diagnosis and/or treatment planning based on the volumetric medical image. The improvement may occur, e.g., compared to conventional (Hounsfield Unit, HU) threshold-based bone identification. The improvement can in particular also apply to medical scans in the presence of a contrast agent. The contrast agent may, e.g., pose problems when applying the conventional HU threshold-based bone identification.

The volumetric medical imaging modality (also three-dimensional, 3D, medical imaging modality) may comprise computed tomography (CT), and/or medical resonance tomography (MRT). Alternatively or in addition, the medical scanner may comprise a CT scanner and/or an MRT scanner. Further alternatively or in addition, the scanner may comprise a combination of medical imaging modalities, such as PET-CT with positron emission tomography (PET) as further medical imaging modality.

The medical scan may be performed on at least one body part (e.g., thorax, head or knee) of a patient. The patient may be a human or an animal (e.g., a mammal, such as a horse or dog).

In some embodiments, the medical scan is performed using a contrast agent. For these images it has been proved that the inventive technique, using in particular a DL metal detection algorithm and a DL MAR algorithm, can provide better quality results than conventional techniques, in particular as conventionally regions with contrast agent are often mistaken as metal or other dense material, such as bone tissue.

The received at least one volumetric image reconstruction may be uncorrected (in particular with respect to metal artifacts; meaning: without metal artifact reduction performed), and/or may comprise metal artifacts (e.g., due to scattering or photoeffect-related beam-hardening events of X-rays at the metal surface in case of a CT scan). MAR algorithms are known to reduce the disturbing effects of metal and provide corrected images.

The at least one volumetric image reconstruction may be specific to a medical question (also: clinical indication), and/or may be protocol-induced. Alternatively or in addition, one or more standard volumetric image reconstructions may be performed based on the body part of the medical scan. Standard volumetric image reconstruction may include, e.g., spectral postprocessing (SPP), such as for virtual monoenergetic images (VMI) and/or virtual non-contrast images, and/or multi-planar reformat (MPR), e.g., sagittal, coronal, and/or axial, and/or with a predetermined slice thickness (e.g., thin or thick).

Alternatively or in addition, the at least one volumetric image reconstruction may comprise multiple volumetric image reconstructions (also: multiple series), each associated with a different medical question and/or adapted to optimize the visibility of a predetermined tissue type, a predetermined organ, and/or a predetermined viewing direction. E.g., a first volumetric image reconstruction of a medical scan of a patient's thorax may be adapted to optimize the visibility of the patient's spine, and/or a second volumetric image reconstruction of the same medical scan may be adapted to optimize the visibility of the lung.

The metal may comprise (or may be comprised in) an implant. The implant may be a dental filling, and/or an endoprosthesis (such as for the hip, knee, shoulder, extremities, and/or comprising a pacemaker, a stent, a spine implant, and/or an endovascular embolization coil). The implant may comprise different metallic regions with different metal types or alloys.

The metal being comprised within data, in particular within the volumetric image reconstruction and/or within any raw data and/or within any projection data (e.g., a sinogram), may denote that the metal is localized within the volume, which is captured (also: imaged) by the medical scan.

The term “metal” within the context of the inventive technique may be interpreted broadly. Any implanted material, which leads to image artifacts due to the material density and/or due to scattering (e.g., of X-rays) during the medical image acquisition may be considered as “metal” in the sense of the invention. E.g., (in particular biocompatible and hard) ceramics used as implants may be comprised as well as conventional biocompatible and hard metals, such as titanium.

The metal detection algorithm may be DL-based. Alternatively or in addition, the metal detection algorithm may comprise an empirical image analysis, and/or may be performed via a neural network, e.g., a convolutional neural network (CNN), a U-Net, and/or a vision transformer (ViT).

The (in particular DL) MAR algorithm for performing the MAR reconstruction may comprise essentially cutting out a region comprising the metal and interpolating among the neighboring regions in the vicinity of the detected metal. Alternatively or in addition, performing the MAR reconstruction may comprise compensating for scattering events at a metal surface, which can, e.g., change the volumetric image in the vicinity of the detected metal (in particular depending on the patient's tissue type in the vicinity of the detected metal). Further alternatively or in addition, performing the MAR reconstruction may comprise superimposing the metal on the interpolated, and/or scattering corrected, volumetric image reconstruction.

The MAR reconstruction may be provided for display at a viewing station. Alternatively or in addition, providing the MAR reconstruction may comprise (e.g., automatically and/or without manual input) saving the MAR reconstruction with a file name according to a predetermined naming scheme. Thereby, confusions and/or a need for manual selection of a file name may be avoided. The automated file name may further improve a clinical workflow.

Receiving the at least one volumetric image reconstruction of the medical scan may comprise receiving (e.g., preprocessed) raw data of the medical scan. The raw data may be (e.g., in the form of) projection data and/or primary thin slice volume data. Alternatively or in addition, receiving the at least one volumetric image reconstruction of the medical scan may comprise reconstructing the raw data to obtain the at least one volumetric medical image reconstruction.

In some embodiments, raw data from the medical scanner are received, and the volumetric image reconstruction is performed in the PACS, cloud and/or some DICOM node. Thereby, the medical scanner can remain completely agnostic in view of the medical question to be answered by the medical scan. Alternatively or in addition, the scanner need not be equipped with extensive computing resources (in particular processing resources) if performing volumetric image reconstructions is moved away.

The method may further comprise a step of receiving (an/or extracting), in particular without a need for manual input, an indication of an existence of metal comprised within the at least one volumetric image reconstruction.

The indication of the existence of metal may be received as a known existence of an implant and/or may be extracted from the patient's electronic health record (EHR) and/or a stored medical report. The indication may trigger the metal detection algorithm. Receiving or extracting the indication may be executed algorithmically. Alternatively or in addition, receiving the indication may be performed by processing (a in particular simple) user input received on a user interface (e.g., only comprising a check box or confirmation that the metal detection algorithm is to be performed).

Without the indication of the existence of metal, in some embodiments, no MAR reconstruction is performed. Thereby, computational resources (e.g., comprising processing and/or memory resources) can be allocated as necessary, and/or a computational load can be reduced for medical scans without metal.

The metal detection algorithm may comprise a DL metal detection algorithm. Alternatively or in addition, the metal detection algorithm may comprise performing a neural network-based metal detection. Optionally, the neural network may comprise a CNN, in particular a U-Net, and/or a ViT. Alternatively or in addition, the metal detection algorithm may comprise performing a neural network-based metal segmentation (and/or obtaining a, in particular binary, metal mask). Optionally, the neural network may comprise a CNN, in particular a U-Net, and/or a ViT. Further alternatively or in addition, the metal detection algorithm may comprise an empirical image analysis of an image domain, a projection domain, an image's frequency domain, and/or a projection's frequency domain, in particular in view of an existence of a metal within the at least one volumetric image reconstruction.

The metal detection algorithm may use the empirical image analysis in particular for the volumetric image reconstruction (briefly: the reconstruction) of a CT scan. By combining image domain, frequency domain and projection domain, the metal may be detected.

Alternatively or in addition, the metal detection algorithm may be DL-based, and/or may be performed by a neural network, which is in particular suitable for performing a perception task, such as detection, classification, and/or segmentation. The detection, classification, and/or segmentation may comprise classifying metal, bone tissue, soft tissue, and/or air. The neural network may be configured for detecting metal (and/or implants, such as metal objects of known shape and/or of known size, for example based on a list of known implant types). Alternatively or in addition, the neural network may be configured for (in particular semantic) segmentation. E.g., by the segmentation, metal, bone tissue, soft tissue, and/or air may be distinguished.

In case no metal is detected by the (e.g., DL) metal detection algorithm, the inventive technique can stop, as there is no need for performing a MAR reduction on the specific volumetric image reconstruction.

The (in particular DL) MAR algorithm may comprise performing a neural network based metal artifact reduction. Optionally, the neural network comprises a CNN, in particular a U-Net, and/or a ViT. The (in particular DL) MAR algorithm may alternatively or in addition comprise performing a neural network based tissue segmentation and/or air segmentation. Optionally, the neural network for performing (in particular tissue and/or air) segmentation may comprise a CNN, in particular a U-Net, and/or a ViT. Further alternatively or in addition, the (in particular DL) MAR algorithm may comprise an empirical image analysis of an image domain, projection domain, an image's frequency domain, and/or a projection's frequency domain in view of an existence of a patient's tissue (such as soft tissue and/or bone tissue) and/or air (such as in the lungs, airways, and/or background).

Any neural network (e.g., the CNN, such as a U-Net, or a transformer, such as a ViT), e.g., embodying the metal detection algorithm and/or the MAR algorithm, may comprise an encoder and a decoder and/or may perform a predetermined number of downsampling steps for extracting features, followed by a number up upsampling steps.

In some embodiments, two independent neural networks may be applied, one for detecting the metal and providing a metal mask, and a second neural network for segmenting a patient's tissue, such as bone tissue, soft tissue, and/or air, and/or regions with contrast agent. In an alternative embodiment, the same neural network may be applied for providing the metal mask and for providing masks for tissue, air, and optionally a contrast agent.

By the metal detection algorithm, a position, an orientation, dimensions, material, and/or a shape of the metal may be determined. Thereby, the subsequent MAR reconstruction may be enabled and/or improved. E.g., regions to exclude from an interpolation-correction (briefly also: interpolation) may be identified. E.g., if a stent is detected, a volume enclosing the stent may be excluded from the interpolation-correction. Alternatively or in addition, by the interpolation-correction, the blood vessel without the stent may be reconstructed. Alternatively or in addition, by the knowledge about the metal (such as its position and/or shape), image distortions due to noise, such as scattering events during the acquisition of the medical scan (e.g., distortions in regions of the patient's tissue not overlapping with the metal), can be compensated in the subsequent MAR reconstruction.

The steps of detecting the metal, of performing the metal artifact reduction, and/or of providing the MAR at least one volumetric image reconstruction may be performed responsive to the receiving of the at least one volumetric image reconstruction from the scanner. Alternatively or in addition, the steps of detecting the metal, performing the metal artifact reduction, and/or providing the MAR at least one volumetric image reconstruction may be performed responsive to a user's worklist including assessing the medical scan. Further alternatively or in addition, the steps of detecting the metal, performing the metal artifact reduction, and/or providing the MAR at least one volumetric image reconstruction may be performed responsive to a user (in particular a medical expert, such as a radiologist) accessing the received of the at least one volumetric image reconstruction, and/or a case comprising the received of the at least one volumetric image reconstruction. The event based (e.g., based on the user's worklist and/or the user accessing the uncorrected volumetric image reconstruction, and/or accessing a case comprising the uncorrected volumetric image reconstruction) metal detection and MAR reconstruction may in particular apply to a patient's prior imaging, which is retrieved from the PACS for comparison with the patient's most recent medical imaging. Alternatively or in addition, the event based metal detection and MAR reconstruction may be performed for prior studies of other patients used for comparison with a patient's current medical imaging.

In the presence of sufficient computing resources (e.g., sufficient permanent storage), the MAR reconstruction may be performed for any received uncorrected volumetric image reconstruction. Alternatively or in addition, permanent storage may be saved by only temporarily storing (and/or caching) the MAR reconstructions as necessary, e.g., when it is foreseen by the user's (e.g., radiologist's) worklist that he will shortly review the medical scan, and/or when the user accesses the patient's case file and/or the medical scan. Performing the MAR reconstruction based on the worklist may be suitable in case of limited processing power (e.g., spacing out processing such as overnight and/or including weekends, such that the MAR reconstruction is available by a scheduled time according to the user's worklist) and/or in case of a slow MAR reconstruction speed. Performing the MAR reconstruction upon the user accessing the case file, and/or upon accessing the medical scan, may be suitable in case of a high available processing power and/or a fast MAR reconstruction speed.

The steps of detecting the metal, of performing the metal artifact reduction, and/or of providing the MAR at least one volumetric image reconstruction may be performed within a PACS. Alternatively or in addition the steps of detecting the metal, of performing the metal artifact reduction, and/or of providing the MAR at least one volumetric image reconstruction may be performed at a DICOM node. Alternatively or in addition, the steps of detecting the metal, of performing the metal artifact reduction, and/or of providing the MAR at least one volumetric image reconstruction may be performed at a (in particular computing) platform, such as on a local computing device (and/or a, in particular local, workstation and/or a, in particular local, viewing station) and/or an edge (e.g., computing) platform. The platform may be arranged (and/or or located) outside of the scanner pertaining to the volumetric medical imaging modality (briefly also: the scanner, and/or the modality). Further alternatively or in addition, the steps of detecting the metal, of performing the metal artifact reduction, and/or of providing the MAR at least one volumetric image reconstruction may be performed in a computing cloud, and/or at a cloud server. Further alternatively or in addition, the PACS may be hosted by the cloud, and/or the cloud server.

The cloud server (in short: the cloud) may be accessed between a DICOM node (also: DICOM router) and the PACS. Alternatively or in addition, the cloud may be accessed between the PACS and a viewing system.

Performing the MAR reconstruction in the cloud may have the advantage of vast available processing resources.

Performing the MAR reconstruction in the PACS, and/or at a DICOM node (e.g., at a viewing station), may have the advantage of allowing for temporarily available MAR reconstructions only without permanent storing, reducing a need for permanent data storage space (also: memory space).

The step of detecting the metal may be performed independently from the step of performing the metal artifact reduction. E.g., detecting the metal may in some embodiments be performed responsive to receiving the at least one volumetric image reconstruction, and/or performing the metal artifact reduction may be performed at a later time based on the user's worklist and/or based on the user accessing the case file. Alternatively or in addition, the steps of detecting the metal and of performing the metal artifact reduction may be performed by different entities, e.g., one in the cloud, and the other in the PACS (or one at a DICOM node and the other in the cloud or in the PACS), and vice versa.

In case the metal detecting is performed independently, a digital file comprising the at least one volumetric image reconstruction may comprise an indication of the result of the metal detecting. E.g., the indication may be stored as metadata.

The method may further comprise a step of storing the provided MAR at least one volumetric image reconstruction.

Storing may comprise permanent (and/or long-term) storing (also: saving) the MAR reconstruction along with the uncorrected reconstruction. Alternatively or in addition, storing the MAR reconstruction may comprise providing a file name automatically and/or according to a predetermined scheme.

By storing the MAR reconstructions according to a predetermined scheme, retrieval of the MAR reconstruction as necessary can be simplified. The simplification may comprise automatic retrieval and/or manual retrieval by a user (e.g., radiologist and/or oncologist).

The method may further comprise a step of receiving a viewing request in relation to the at least one volumetric image reconstruction. The viewing request may be received at a viewing station and/or at a UI, in particular at a GUI.

Alternatively or in addition, the method may further comprise a step of displaying an (e.g., two-dimensional, 2D) image of the received at least one volumetric image reconstruction and/or an (e.g., 2D) image of the MAR at least one volumetric image reconstruction. Optionally, displaying the image of the received at least one volumetric image reconstruction, and/or displaying the image of the MAR at least one volumetric image reconstruction, may comprise the user selecting an image view (and/or a hanging protocol) of the received at least one volumetric image reconstruction. The corresponding image view (and/or a hanging protocol) of the MAR at least one volumetric image reconstruction may be displayed side-by-side and/or overlayed with the image view (and/or a hanging protocol) of the received at least one volumetric image reconstruction.

The corresponding image view (and/or a hanging protocol) may comprise an identical viewing depth and/or identical viewing direction.

The overlay may be performed with variable, and/or user selected, (e.g., relative) transparencies (and/or intensities) of the uncorrected and the MAR corrected reconstructions.

In some embodiments, the display may be configured for highlighting differences between the uncorrected volumetric image reconstruction and the MAR corrected reconstruction.

The UI may be a GUI.

The user may need to only select the uncorrected reconstruction for viewing. The corresponding MAR reconstruction may be automatically provided. Alternatively or in addition, a hanging protocol in relation to the MAR reconstruction may be automatically derived from the hanging protocol selected, by the user, for the uncorrected reconstruction. Thereby, the user's workflow may be improved.

The volumetric medical imaging modality may comprise CT.

Reducing metal artifacts may be of particular importance when X-rays are used for image acquisition. E.g., scattering of X-rays may introduce significant artifacts in the medical scan.

Performing the metal artifact reduction may comprise performing a (e.g., normalized) representation of a projection domain interpolation between regions of the at least one volumetric image reconstruction that do not comprise the detected metal. Performing the metal artifact reduction may further comprise superimposing the detected metal onto the interpolated at least one volumetric image reconstruction.

Alternatively or in addition, performing the metal artifact reduction may comprise a substep of determining at least one prior image. The at least one prior image may comprise regions of the at least one volumetric image reconstruction without detected metal. Preferably, determining the at least one prior image may be based on a multi-mask segmentation. The multi-mask may comprise one or more air segmentation masks (briefly: air masks), one or more tissue segmentation masks (briefly: tissue masks), and optionally a contrast agent segmentation mask (briefly: contrast agent mask).

Performing the metal artifact reduction may alternatively or in addition comprise a substep of performing a (optionally normalized) interpolation between regions of the at least one volumetric image reconstruction that do not comprise the detected metal. Performing the interpolation may use a representation of a projection domain. Preferably, performing the interpolation is based on projected representations of the at least one volumetric image reconstruction, the at least one prior image and the detected metal.

The prior image may in some embodiments only comprise the multi-mask and thus only provide information on how to implement the interpolation and MAR reduction depending on tissue types (e.g., bone) and a presence of air. In other embodiments, the prior image may comprise regions of the volumetric image reconstruction, in particular in addition to the multi-mask.

Bone tissue (and/or any other tissue type), as well as the voxels detected by the corresponding (in particular bone, and/or any other tissue type) mask (and/or the segmentation mask) may be used 1:1 in the prior image. Analogously, air and/or the voxels detected by an air mask (and/or the segmentation mask) may be used 1:1 in the prior image.

Performing the metal artifact reduction may alternatively or in addition comprise a substep of superimposing the detected metal onto the at least one volumetric image reconstruction and/or onto the interpolated at least one volumetric image reconstruction. Preferably, the superimposing comprises modifying a low-frequency part of the at least one volumetric image reconstruction by the performed interpolation between regions that do not comprise the detected metal. Modifying the low-frequency part of the at least one volumetric image reconstruction may also be denoted as performing a frequency split.

The representation of the projection domain may also be denoted as sinogram. Alternatively or in addition, the representation of the projection domain interpolation (also: interpolation of the representation of the projection domain) may briefly be denoted as sinogram interpolation.

Normalizing the interpolation of the representation of the projection domain (also: sinogram interpolation) can help in reducing the metal artifacts in the reconstruction.

Before performing the superimposing, on the interpolated reconstruction without the metal, a denormalizing step may be performed.

The interpolation may be performed only on a local region (e.g., comprising a predefined distance from the detected metal) of the uncorrected volumetric image reconstruction.

An uncorrected sinogram may be normalized voxel-wise towards the tissue classes (e.g., bone, air, and/or soft-tissue), such as described above as tissue-type specific segmentation, before the sinogram interpolation. The steps may be: firstly performing the sinogram normalization (e.g., the original sinogram is divided by the tissue-sinogram, or “prior” sinogram). Secondly, a sinogram interpolation to reduce metal artifacts may be performed. Thirdly, a sinogram denormalization (e.g., normalized-interpolated sinogram, multiplied by the same tissue sinogram as used in the first step for the normalization) may be performed.

Sinogram interpolation may, e.g., describe the modification of a 2D parallel-beam sinogram by modifying values of the metal trace, such as all projections, which include metal and/or scattering (or other noise) related to the presence of the metal.

Metal projections may be acquired from forward-projecting of a (e.g., binarized) metal mask, such as obtained by the neural network for metal detection.

The at least one prior image may comprise a multi-mask. The multi-mask may comprise a mask for each of the patient's tissue type (e.g., bone tissue and soft tissue, and optionally further refined depending on the specific bone type and/or soft tissue type), a mask for air in the lungs and/or in the airways, a mask for background air, and/or a mask for regions, where a contrast agent is present. The mask for the contrast agent may specify the type of contrast agent.

For obtaining the MAR reconstruction, a (in particular image-based) frequency split may be applied after the sinogram interpolation (and/or the interpolation of the representation of the projection domain). Thereby, anatomical details of the medical scan may be restored.

The frequency split may comprise retaining high frequency contributions to the volumetric image reconstruction from the uncorrected volumetric image reconstruction. Alternatively or in addition, the frequency split may comprise modifying low frequency contributions to the volumetric image reconstruction by using the (in particular normalized) interpolation in the projection domain (also: of the projection data).

The frequency contributions to the volumetric image reconstruction may comprise different frequency-split limits.

Frequencies in an image or projection may in general be extracted by Fourier analysis and/or may serve to describe how neighboring pixels' values change.

The normalized sinogram interpolation step may be performed based on techniques described in [5] or [7]. Alternatively or in addition, the step of applying the frequency split may be based on techniques described in [9] and [10].

The method may further comprise a step of providing a text draft for inclusion in a medical report on the medical scan. The method may further comprise a step of receiving a user input in view of the provided text draft. Optionally, the user input comprises an acceptance, a rejection, or a modification of the (in particular automatically generated and/or provided) text draft.

By providing the text draft for the medical report, the user may be further supported in an efficient workflow. Alternatively or in addition, omissions of potentially relevant findings within the medical scan may be reduced. An accuracy of the medical report may thereby be increased.

In some embodiments, the text draft may further comprise, and/or may be based, on a medical history of a patient's body part for which the medical scan was performed.

The metal detection algorithm and/or the (in particular DL) MAR algorithm may be trained using (in particular per slice, such as axial thin slice) annotated training data. Preferably, the annotated training data may comprise, as ground truth, at least a metal segmentation mask for the metal detection algorithm and (e.g., bone and/or soft) tissue segmentation masks, (e.g., lung and airways and/or background) air segmentation masks, and optionally a (e.g., contrast agent-specific) contrast agent segmentation mask for the MAR algorithm.

The metal detection algorithm may be trained based on annotated training data comprising (in particular manually) provided metal masks associated with volumetric image reconstructions of medical scans.

The (in particular DL) MAR algorithm may be trained based on annotated training data comprising tissue masks, air masks, and optionally contrast agent masks, associated with volumetric image reconstructions of medical scans. Preferably, the tissue masks comprise soft tissue masks and bone tissue masks.

Optionally, the metal detection algorithm and the (e.g., DL) MAR algorithm are jointly trained based on annotated training data comprising the metal masks, the tissue masks, the air mask, and optionally the contrast agent masks.

As to a device aspect, a computing device for providing a MAR volumetric image reconstruction is provided. The computing device comprises a first interface. The first interface is configured for receiving at least one volumetric image reconstruction of a medical scan from a scanner pertaining to a volumetric medical imaging modality. The computing device further comprises a metal detection module configured for detecting, via a metal detection algorithm, a metal comprised within the at least one volumetric image reconstruction. The computing device further comprises a metal artifact reduction module configured for performing, responsive to the detecting of the metal and via a (in particular DL) MAR algorithm, a metal artifact reduction on the received at least one volumetric image reconstruction. The computing device further comprises a second interface configured for providing the MAR at least one volumetric image reconstruction.

The first interface of the computing device may be configured for receiving raw data of the medical scan, in particular raw data in form of projection data and/or primary thin slice volume data. The computing device may comprise an image reconstruction module configured for reconstructing the raw data to obtain the at least one volumetric medical image reconstruction.

The metal artifact reduction module of the computing device may comprise a prior image determination submodule, which is configured for determining at least one prior image. The at least one prior image may comprise regions of the at least one volumetric image reconstruction without detected metal. Preferably, determining the at least one prior image is based on a multi-mask segmentation (e.g., comprising one or more air masks, one or more tissue masks, and optionally one or more contrast agent masks).

The metal artifact reduction module of the computing device may alternatively of in addition comprise an interpolation submodule configured for performing a (e.g., normalized) interpolation between regions of the at least one volumetric image reconstruction that do not comprise the detected metal using a representation of a projection domain (also: projection representation, and/or projection data, e.g., a sinogram). Preferably, performing the interpolation may be based on projected representations of the at least one volumetric image reconstruction, the at least one prior image and the detected metal.

The metal artifact reduction module of the computing device may further alternatively or in addition comprise a superposition submodule (also denoted as frequency-split submodule), which is configured for superimposing the detected metal onto the at least one volumetric image reconstruction and/or onto the interpolated at least one volumetric image reconstruction. Preferably, the superimposing may comprise modifying a low-frequency part of the at least one volumetric image reconstruction by the performed interpolation between regions that do not comprise the detected metal.

The computing device may further comprise a third interface (and/or an indication extraction module) for receiving (and/or for extracting) an indication of an existence of a metal comprised within the at least one volumetric image reconstruction.

The computing device may further comprise memory configured for storing the provided MAR at least one volumetric image reconstruction.

The computing device may further comprise a fourth interface (in particular a UI, such as a GUI) configured for receiving a viewing request in relation to the at least one volumetric image reconstruction.

The computing device may further comprise a fifth interface (e.g., a GUI) configured for displaying an image of the received at least one volumetric image reconstruction and/or an image of the MAR at least one volumetric image reconstruction. Optionally, displaying the image of the received at least one volumetric image reconstruction and/or displaying the image of the MAR at least one volumetric image reconstruction may comprise that the user selects an image view of the received at least one volumetric image reconstruction, and the corresponding image view of the MAR at least one volumetric image reconstruction is displayed side-by-side, and/or overlayed, with the image view of the received at least one volumetric image reconstruction.

The computing device may further comprise a draft provision module (and/or a sixth interface) configured for providing a text draft for inclusion in a medical report on the medical scan.

The computing device may further comprise a seventh interface configured for receiving a user input in view of the provided text draft. Optionally, the user input may comprise an acceptance, a rejection, or a modification of the in particular automatically generated and/or provided text draft.

The computing device may be configured to perform any one of the steps, or comprise any one of the features, described in the context of the method aspect.

As to a system aspect, a system for providing a MAR volumetric image reconstruction is provided. The system comprises at least one scanner pertaining to a volumetric medical imaging modality. The system further comprises at least one computing device according to the device aspect. Alternatively or in addition, the system may comprise a computing cloud and/or a cloud server. The first interface of the computing device may be configured for receiving the at least one volumetric image reconstruction of a medical scan from the at least one scanner. Alternatively or in addition, the at least one volumetric image reconstruction of a medical scan may be received by the cloud server, and/or in the cloud, from the at least one scanner. Optionally, the system may comprise at least one GUI (e.g., integrated in a viewing station). The GUI may be configured for displaying the received at least one volumetric image reconstruction and the MAR at least one volumetric image reconstruction.

The system may be configured to perform any one of the steps, or comprise any one of the features, described in the context of the method aspect.

As to a further aspect, a computer program product is provided, which comprises program elements which induce a computing device (e.g., the computing device according to the device aspect) to carry out the steps of the method for providing a MAR volumetric image reconstruction according to the method aspect, when the program elements are loaded into a memory of the computing device.

As to a still further aspect, a computer-readable medium is provided, on which program elements are stored that can be read and executed by a computing device (e.g., the computing device according to the device aspect), in order to perform steps of the method for providing a MAR volumetric image reconstruction according to the method aspect, when the program elements are executed by the computing device.

This following description does not limit the invention on the contained embodiments. Same components or parts can be labelled with the same reference signs in different figures. In general, the figures are not for scale.

It shall be understood that a preferred embodiment of the present invention can also be any combination of the dependent claims or above embodiments with the respective independent claim.

These and other aspects of the invention will be apparent from and elucidated with reference to the embodiments described hereinafter.

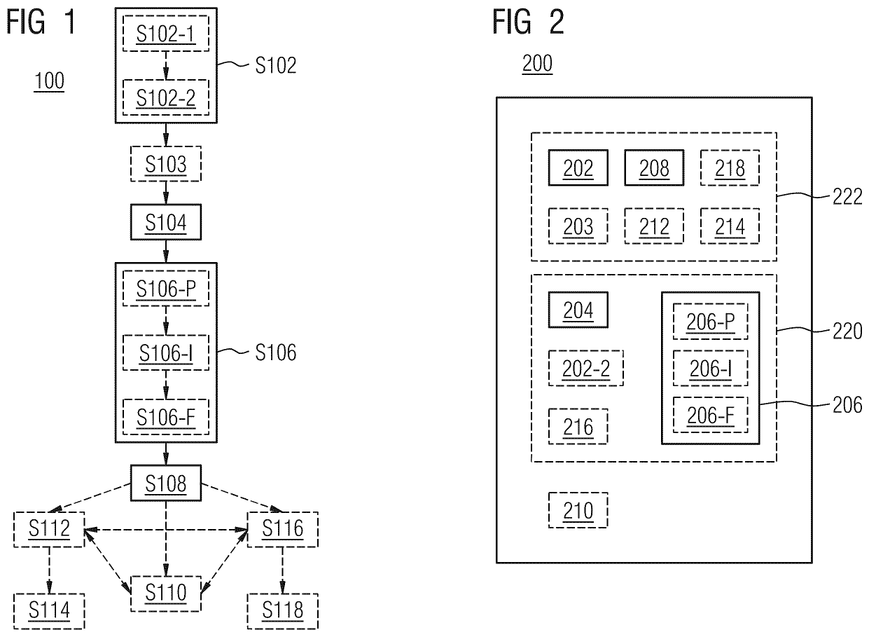

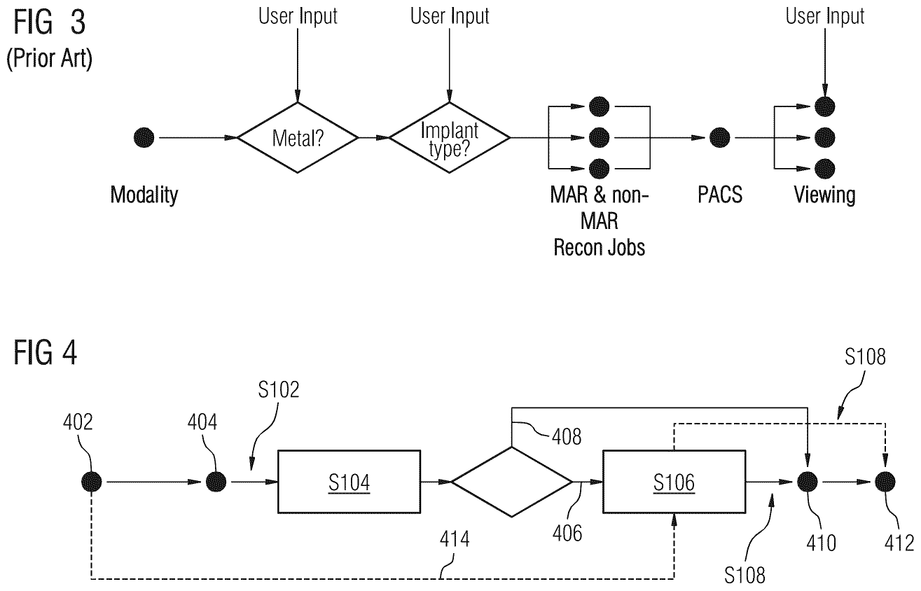

FIG. 1 schematically illustrates an exemplary flowchart for a (in particular computer-implemented) method for providing a metal artifact reduced (MAR) volumetric image reconstruction. The method is generally referred to by the reference sign 100.

The method 100 comprises a step S102 of receiving at least one volumetric image reconstruction of a medical scan from a scanner pertaining to a volumetric medical imaging modality. The step of receiving S102 the at least one volumetric image reconstruction of the medical scan may comprise a sub-step S102-1 of receiving raw data of the medical scan (in particular raw data in form of projection data and/or primary thin slice volume data) and a sub-step S102-2 of reconstructing the raw data to obtain the at least one volumetric medical image reconstruction.

The method 100 further comprises a step S104 of detecting a metal comprised within the at least one volumetric image reconstruction via a metal detection algorithm.

The method 100 further comprises a step S106 of performing a metal artifact reduction (MAR) on the received S102 at least one volumetric image reconstruction responsive to the detecting S104 of the metal and via a, in particular deep learning (DL), MAR algorithm. The step S106 of performing the metal artifact reduction may comprise a substep S106 of determining at least one prior image. The prior image may comprise regions of the at least one volumetric image reconstruction without detected metal. Preferably, determining S106-P the at least one prior image is based on a multi-mask segmentation, the multi-mask comprising one or more air masks (e.g., a mask for air of the background and a further mask for air inside the human body, in particular within the airways and lung), one or more tissue masks (e.g., a mask for soft tissue, such as muscle, fat, and/or inner organs, and/or a mask for bone tissue), and optionally a contrast agent mask (e.g., if there is an indication, such as comprised in metadata of the received at least one volumetric image reconstruction). The step S106 may further comprise a substep S106-I of performing an interpolation of a (e.g., normalized) representation of a projection domain, which interpolates between regions of the at least one volumetric image reconstruction that do not comprise the detected S104 metal. The interpolating S106-I may use a representation of a projection domain. Preferably performing S106-I the interpolation is based on projected representations of the at least one volumetric image reconstruction, the at least one prior image and the detected S104 metal (e.g., a metal mask). The step S106 may further comprise a substep S106-F of superimposing the detected S104 metal onto the at least one volumetric image reconstruction and/or onto the interpolated at least one volumetric image reconstruction. Preferably, the superimposing S106-F comprises modifying a low-frequency part of the at least one volumetric image reconstruction by the performed S106-I interpolation between regions that do not comprise the detected S104 metal.

The method 100 further comprises a step S108 of providing the MAR at least one volumetric image reconstruction.

Optionally, the method 100 comprises a step S103 of receiving (or extracting, e.g., from an EHR for the patient to whom the medical scan belongs) an indication of an existence of a metal comprised within the at least one volumetric image reconstruction.

The method 100 may comprise a step S110 of storing the provided S108 MAR at least one volumetric image reconstruction.

The method 100 may comprise a step S112 of receiving a viewing request in relation to the at least one volumetric image reconstruction, in particular at a user interface (UI) such as a graphical user interface (GUI). The method 100 may further comprise a step S114 of displaying an image of the received S102 at least one volumetric image reconstruction and/or displaying an image of the MAR at least one volumetric image reconstruction. Optionally, displaying the image of the received S102 at least one volumetric image reconstruction and/or displaying the image of the MAR at least one volumetric image reconstruction may comprise the user selecting an image view of the received S102 at least one volumetric image reconstruction, and the corresponding image view of the MAR at least one volumetric image reconstruction may be displayed side-by-side, or overlayed, with the image view of the received S102 at least one volumetric image reconstruction, in particular in an at least semi-automated manner.

Any step of displaying an image and/or of a user interaction, or user feedback, may be performed at a viewing station, which may comprise the UI, such as a GUI. User inputs may alternatively or in addition be received by any other input means (and/or UIs) such as a keyboard, mouse, and/or trackpad.

The method 100 may comprise a step S116 of providing a text draft for inclusion in a medical report on the medical scan. The method 100 may further comprise a step S118 of receiving a user input in view of the provided text draft. Optionally, the user input comprises an acceptance, a rejection, or a modification of the in particular automatically generated and/or provided text draft.

The step S110 of storing the provided S108 MAR at least one volumetric image reconstruction may be performed before or after (and/or independently of) the step S112 of receiving a viewing request and/or the step S116 the text draft. Alternatively or in addition, the text draft may be provided S116 before or after (and/or independently of) the step S112 of receiving the viewing request.

According to some embodiments, the step S104 of detecting the metal, the step S106 of performing the metal artifact reduction and the step S108 of providing the MAR at least one volumetric image reconstruction are triggered (and/or only performed) by the receiving S112 of the viewing request.

FIG. 2 schematically illustrates an exemplary architecture of a computing device for providing a MAR volumetric image reconstruction. The computing device is generally referred to by the reference sign 200.

The computing device 200 comprises a first interface 202, which is configured for receiving at least one volumetric image reconstruction of a medical scan from a scanner pertaining to a volumetric medical imaging modality. The computing device 200 further comprises a metal detection module 204, which is configured for detecting, via a metal detection algorithm, a metal comprised within the at least one volumetric image reconstruction. The computing device 200 further comprises a metal artifact reduction module 206, which is configured for performing, responsive to the detecting of the metal and via a (in particular DL) MAR algorithm, a metal artifact reduction on the received at least one volumetric image reconstruction. The computing device 200 further comprises a second interface 208, which is configured for providing the MAR at least one volumetric image reconstruction.

The first interface 202 may be configured for receiving raw data of the medical scan, such as raw data in form of projection data and/or primary thin slice volume data. The computing device 200 may further comprise an image reconstruction module 202-2, which is configured for reconstructing the raw data to obtain the at least one volumetric medical image reconstruction.

The metal artifact reduction module 206 may comprise a prior image determination submodule 206-P, which is configured for determining at least one prior image. The at least one prior image comprises regions of the at least one volumetric image reconstruction without detected metal. Preferably, determining the at least one prior image is based on a multi-mask segmentation, the multi-mask comprising one or more air masks, one or more tissue masks, and optionally a contrast agent mask.

The metal artifact reduction module 206 may further comprise an interpolation submodule 206-I configured for performing a (optionally normalized) interpolation between regions of the at least one volumetric image reconstruction that do not comprise the detected metal using a representation of a projection domain. Preferably, performing the interpolation is based on projected representations of the at least one volumetric image reconstruction, the at least one prior image and the detected metal.

The metal artifact reduction module 206 may comprise a superposition submodule (also denoted as frequency-split submodule) 206-F, which is configured for superimposing the detected metal onto the at least one volumetric image reconstruction and/or onto the interpolated at least one volumetric image reconstruction. Preferably, the superimposing comprises modifying a low-frequency part of the at least one volumetric image reconstruction by the performed interpolation between regions that do not comprise the detected metal.

The computing device 200 may comprise a third interface 203 (or an indication extraction module, not shown in FIG. 2), which may be configured for receiving (or extracting) an indication of an existence of a metal comprised within the at least one volumetric image reconstruction.

The computing device 200 may comprise a memory 210, which is configured for storing the provided MAR at least one volumetric image reconstruction. The memory 210 may alternatively or in addition be configured for storing computer program code for performing the method 100. Further alternatively or in addition, the memory 210 may be configured for storing intermediate results of performing the method 100.

The computing device 200 may comprise a fourth interface 212, which is configured for receiving (e.g., from a UI, such as a GUI) a viewing request in relation to the at least one volumetric image reconstruction.

The computing device 200 may comprise a fifth interface 214, which is configured for displaying (e.g., on the GUI) an image of the received at least one volumetric image reconstruction and/or an image of the MAR at least one volumetric image reconstruction. Optionally, displaying the image of the received at least one volumetric image reconstruction and/or displaying the image of the MAR at least one volumetric image reconstruction may comprise that the user selects an image view of the received at least one volumetric image reconstruction, and the corresponding image view of the MAR at least one volumetric image reconstruction is displayed side-by-side, or is overlayed, with the image view of the received at least one volumetric image reconstruction.

The computing device 200 may comprise a draft provision module 216 (and/or a sixth interface, not shown in FIG. 2), which is configured for providing a text draft for inclusion in a medical report on the medical scan. The computing device 200 may further comprise a seventh interface 218, which is configured for receiving a user input in view of the provided text draft. Optionally, the user input may comprise an acceptance, a rejection, or a modification of the in particular automatically generated and/or provided text draft.

The computing device 200 may comprise a processor 220. The processor 220 may embody the metal detection module 204, the metal artifact reduction module 206, the optional image reconstruction module 202-2, the optional draft provision module 216, and/or the optional indication extraction module.

The computing device 200 may comprise an input-output interface 222. The input-output interface 222 may embody any one of the interfaces (e.g., the interfaces shown at reference signs 202; 203; 208; 212; 214; 218 in FIG. 2, and/or the optional sixth interface).

The computing device 200 may be configured for performing the method 100.

A system for providing a MAR volumetric image reconstruction may comprise at least the computing device 200 and at least one scanner pertaining to a volumetric medical imaging modality, with the first interface 202 of the computing device 200 being configured for receiving the at least one volumetric image reconstruction of a medical scan from the at least one scanner. Optionally, the system may further comprise at least one GUI, which is configured for displaying the received at least one volumetric image reconstruction and the MAR at least one volumetric image reconstruction provided by the at least one computing device 200.

The system may be configured to perform the method 100.

The inventive technique (e.g., comprising the method 100, and/or the computing device 200) may alternatively be denoted as PACS- and/or cloud-based metal artifact reduction framework including a, in particular DL-based, metal artifact detection method.

While in the following, embodiments are mainly directed to CT and/or X-Ray-based images, a workflow may be equally applicable to a metal artifact correction or other artifact correction in MRT images. In particular, workflow-related benefits described in the context of the inventive technique are generally applicable to artifacts in medical images and their reduction and/or correction.

DL-based methods can be used to address multiple drawbacks within the conventional imaging chain.

Generally, a scanner provides raw data (and/or measurement data) which may be pre-processed to provide a projection representation, such as sinogram (and/or, in particular 2D, matrix structure) for a CT scan. The raw data of a CT scan may, e.g., comprise X-ray intensities detected after passing through the patient.

The sinogram may be the basis for image reconstruction. A sinogram may be understood as a preprocessed form of the raw data of the scanner. Thus, a sinogram is a raw data representation of a CT scan before image reconstruction. It contains the X-ray projection data collected from multiple angles as the scanner rotates around the patient. Each row in the sinogram may correspond to a specific detector element. Each column may represent projections taken from different angles. Alternatively or in addition, a single point object inside the scanned region (in particular if the scan covers more than 180 degree) may typically appear as a sine wave in the sinogram.

For CT image reconstruction from raw data, however, different methods may be applied, which do not (or may not) necessarily involve providing a sinogram. For example, conventionally, filtered backpropagation (FBP) may be used. In FBP, after the raw data is collected from multiple angles, a mathematical filter is applied to correct blurring and adjust an image impression, and finally a back-projection is performed to reconstruct the image slice-by-slice. Alternatively or in addition, iterative reconstruction (IR) is an advanced technique that iteratively refines the image to improve quality while reducing noise and artifacts. In a simplified manner, IR may be described to start with an image (possibly, but not limited to, from FBP) as initial image. In a so-called forward projection, synthetic projections based on the estimated image are simulated. The system identifies discrepancies between the synthetic and actual raw data and updates the image by reducing inconsistencies. This process is repeated iteratively until the image stabilizes. The IR-reconstructed image serves then as the input for the inventive MAR algorithm.

An advantage of the inventive technique is that it is independent of the way the volumetric image reconstruction was performed. By basing the MAR (in particular solely) on the received volumetric image reconstruction and performing the metal artifact reduction on the PACS, at a DICOM node, and/or in a computing cloud, it becomes independent of the manufacturer of the scanner, by which the medical (e.g., CT) scan was obtained (which may also be denoted as vendor-unspecific technique). Alternatively or in addition, the inventive technique may provide a MAR image reconstruction based on volumetric images and/or tomographic images (and/or image slices) generated by various reconstruction techniques, such as IR-generated and/or FBP-generated (in particular weighted FBP, WFBP, generated) volumetric images and/or tomographic images (and/or image slices).

FIG. 4 shows an exemplary embodiment of the inventive technique (e.g., comprising the method 100, and/or the computing device 200), which starts with the medical scan at the scanner (also: modality) at reference sign 402. At reference sign 404, the volumetric image reconstruction (Recon), also denoted as “normal Recon Jobs” is performed.

The inventive technique in the exemplary embodiment in FIG. 4 includes both a DL-based metal artifact detection system (in particular for performing the step S104) and a retrospective DL- and image-based MAR algorithm (in particular for performing the step S106), both deployed in either the PACS or a cloud-based image-handling framework. In FIG. 4, further detailed is shown at the diamond that, at reference sign 408, if no metal is detected, the method (also: pipeline) continues with forwarding the volumetric image reconstruction to the PACS at reference sign 410. If metal is detected, as indicated at reference sign 406, the step S106 is performed, with two different options of providing the MAR volumetric image reconstruction, namely in one option providing the MAR volumetric image reconstruction to the PACS at reference sign 410, with viewing at reference sign 412 based on the PACS 410. In the other option, the viewing 412 proceeds directly after the retrospective DL-based metal artifact reduction step S106, in particular without a need of going through the PACS 410.

In one embodiment, the method 100 may make use of only the (in particular uncorrected) volumetric image reconstruction received from the scanner. In an alternative embodiment, the method 100 may, along with the (in particular uncorrected) volumetric image reconstruction, make use of the raw data (also: measurement data) acquired by the processor, and/or may make use of preprocessed raw data (e.g., a projection and/or sinogram, based on which the uncorrected volumetric image reconstruction is performed). In FIG. 4, at reference sign 414, an example is indicated, in which the projection data are used (in particular directly) as input to the DL MAR algorithm at reference sign S106. In this case, no projection and/or transformation to obtain an uncorrected projection representation (e.g., sinogram) is required.

According to an exemplary embodiment, the method 100 comprises the following imaging chain: An operator of the scanner (also: CT system) is required to setup the acquisition and reconstruction protocols based on a clinical indication. At this point, according to the inventive technique no additional user input is required to setup metal artifact-related reconstruction tasks. All reconstruction jobs are performed (e.g., as conventionally and/or as before). At this point, according to the inventive technique no additional reconstructions are required for the metal artifact reduction, so only standard reconstructions and protocol-induced additional reconstructions (such as MPRs) need to be performed, sent (in particular from the scanner to the PACS), and stored (e.g., according to the step S110). After sending images from the scanner (also: the modality) to the PACS (e.g., according to the step S102), a metal artifact detection mechanism is used to identify image series, which require additional metal artifact reduction (in particular according to the step S104). This detection mechanism includes several characteristics. A first characteristic may be a deployment scenario. The metal artifact detection tool can, e.g., either be deployed directly on the PACS or in a cloud-based environment in-between the DICOM nodes of the modality and the PACS. A second characteristic may be a triggering mechanism. According to some examples, a detection may be performed for each series and/or study. According to other examples, in order to save computing resources, a detection (e.g., the step S104) need only (or may only) be performed for cases, where there are indications for a metal artifact. This indication may, e.g., come from medical reports of the patient describing implants. Alternatively or in addition, such an indication may be derived from the fact that the data routed to the PACS already comprises corrected series generated at the scanner (also: the modality). A third characteristic may be a method. Multiple approaches to detect the necessity for MAR are feasible. Fully convolutional (FC) networks, CNNs, and/or vision transformers (ViTs) may be deployed to detect metal artifacts by neural networks. Alternatively or in addition, conventional empirical image analysis tools may be used to analyze the image-domain, frequency-domain and projection-domain (and/or sinogram) of the image. Within the sinogram, the metal projections may be identified from forward-projection of a metal mask from the image domain. Then, a frequency analysis of the metal projection space may be used to determine the strength of the metal artifacts and if additional metal artifact reduction is required. A fourth characteristic may be that operationally, it is conceivable that the detection (e.g., the step S104) and correction (e.g., the step S106) are carried out in the background once a case is loaded by a user (e.g., as the step S112 of a viewing request) or based on the worklist of the user. When a case is loaded, the results may be offered to the user in the form of an image enhancement (e.g., similar to google's image enhancing function, and/or as the step S114). This may mean that the user may be provided with a notification that an image enhancement is available. If the user selects to see the enhancements, the corrected images (also: MAR-corrected images, MAR volumetric image reconstructions and/or MAR image reconstructions) may be handled in the viewer as indicated below.

The next steps of the exemplary embodiment and/or the imaging chain may depend on the metal detection framework. If no metal is detected in the volume, no additional steps are required. If metal is detected by the prior metal detection algorithm (in particular in the step S104), an image-based metal artifact reduction is applied (in particular as the step S106) before the volume gets loaded in the PACS (e.g., as the step S108). By that, an online metal artifact detection during image viewing can be performed in real time (e.g., according to the steps S112 and S114). The additional metal artifact reduction series may be stored temporarily, but not shown as a similar series (e.g., similar to GSPS data). Optionally, if the case already contains corrected images which were, e.g., generated by a metal artifact correction at the scanner (also: at the modality), a comparison between the newly generated corrected images and the previously forwarded ones may be made. The comparison may be directed to determine which one has a better image quality. This may be done by standard image quality metrics, such as noise, and/or image artifacts, such as edges. Alternatively or in addition, the quality may be determined by applying computer aided detection tools to both (e.g., the uncorrected and the corrected) images. If, for instance, more findings are detected in the newly corrected image, this may be an indication that the newly corrected image offers a better basis for the ensuing reading and reporting. Optionally, feedback may be given to the scanner (also: modality) about the quality of the correction performed there. If, for instance, a plurality of presets was tested, the best one may be indicated.

In the exemplary embodiment and/or the imaging chain, optionally, the corrected image may be stored S110 together with the original image. If the case already contains corrected images, e.g., coming from the scanner (also: modality), and the newly corrected images are better (e.g., determined using standard image quality metrics and/or image artifacts), the already existing images (in particular stemming from the scanner) may be purged and replaced by the newly generated corrected images (in particular generated by the inventive technique, such as in the PACS, a cloud, and/or at a DICOM node).

In the exemplary embodiment and/or the imaging chain, within the PACS, only the respective uncorrected image series needs to (or must) be selected for viewing, in particular according to the step S112. The uncorrected series can either be dragged manually into the hanging layout, or automatically hanged by the PACS. Then, if the presence of the additional MAR series is detected, a toggle function automatically enables the visualization of the metal artifacts (in particular according to the step S114), and an optional sliding function lets the user interact with the strength of the shown MAR image.

According to some examples, it is suggested to generate an overlay with the corrected and uncorrected image for viewing (e.g., in the step S114). Changes between the (in particular uncorrected and corrected) images may be highlighted, e.g., very much like in the ChangeViz tool. Alternatively or in addition, the user may switch between corrected and uncorrected images (e.g., in the step S114), e.g., like in the MagicLens tool. This provides the benefit that it is simpler for the user to tell if a region is affected by (e.g., MAR-introduced) image artifacts of pathological abnormalities.

Regarding the further workflow of the exemplary embodiment and/or the imaging chain (and/or in a diagnostic cockpit), medical reports may be pre-populated (e.g., according to the step S116) with the information gained with the artifact detection and correction. In some examples, the patient's medical history may be queried for further information regarding an artifact, e.g., based on the body part where the artifact was found.

The inventive technique comprises an AI-based approach to optimize interpolation-based metal artifact reduction, such as in CT.

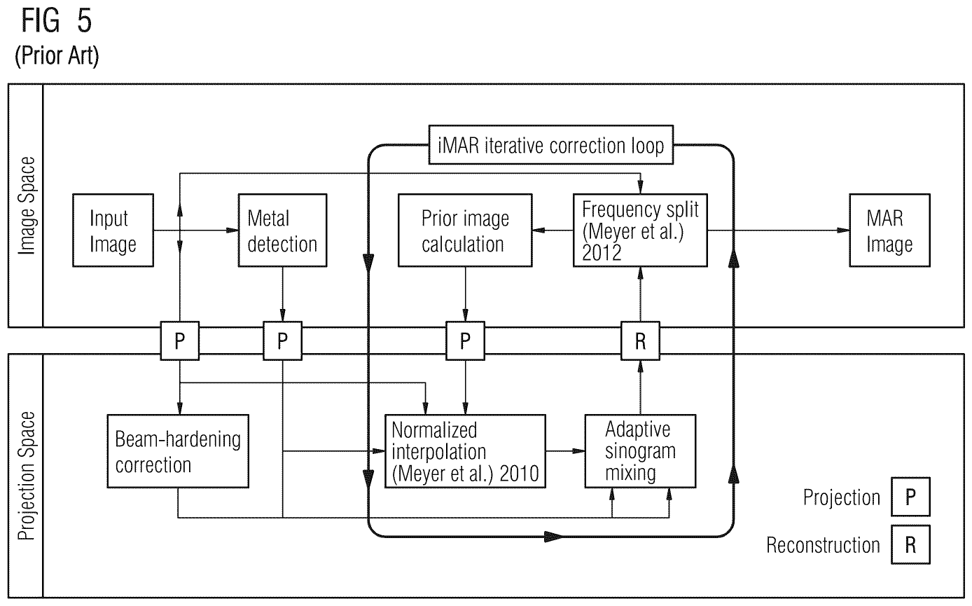

U-Nets, a subtype of convolutional neural networks (CNN), have proven to be powerful tools for image-based segmentation tasks, as described in [8]. The inventive technique for providing a metal artifact reduced volumetric image reconstruction (and/or for an enhanced metal artifact reduction framework) introduces in some embodiments the application of a U-Net, or another CNN, to replace the conventionally used thresholding steps (e.g., as sown in FIG. 5) to calculate a more precise estimation of the prior image (and/or, in particular uncorrected, volumetric image reconstruction of a medical scan), which is used, e.g., for NMAR's sinogram interpolation.

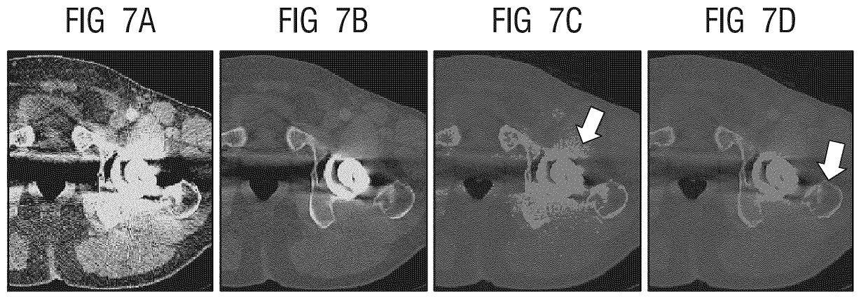

The U-Net may be applied to calculate regions of bone, air, and/or soft tissue. By that, the effect of including wrong areas within the prior image (e.g., as shown in FIGS. 7A to 7D) is reduced. This directly effects the image quality after metal artifact reduction. To further reduce the need of requiring an additional user input, the conventionally used threshold-based metal segmentation may be replaced by a second U-Net or another CNN.

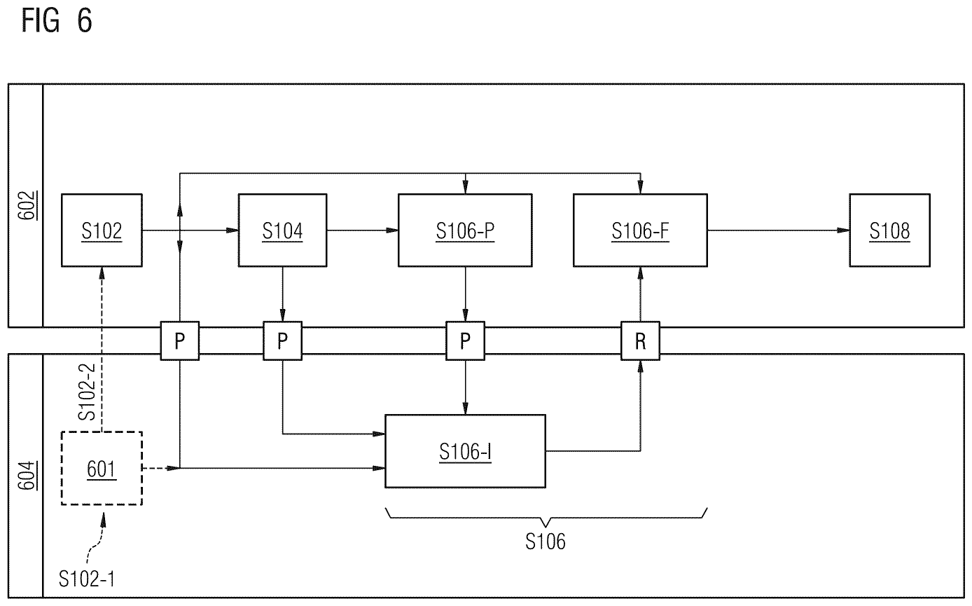

FIG. 6 schematically illustrates an embodiment of the inventive technique for providing a metal artifact reduced volumetric image reconstruction (and/or for metal artifact reduction), which replaces the conventional steps of manually thresholding Hounsfield Unit (HU) values by applying a neural network. Those neural networks may be used to calculate, e.g., both the segmentation masks of air and bone within the prior image. In addition, to reduce the need for user-selectable presets, a second neural network may be applied to calculate the metal mask.

The upper half of FIG. 6 operates in the image space as indicated at reference sign 602. The lower half of FIG. 6 operates in the projection space as indicated at reference sign 604. As indicated at reference sign S106-P, a DL-based prior image calculation may be performed in the image space. A normalized interpolation, e.g., similar to the normalized interpolation described in [5] or [7], is performed based on the determined (also: calculated) metal mask (e.g., in the step S104) and the determined prior image at reference sign S106-P. At reference sign S106-F, a frequency split, e.g., similar to the frequency split described in [9], is performed. The normalized interpolation and frequency split at reference signs S106-I and S106-F, respectively may be comprised in the method step S106 of performing the metal artifact reduction to arrive at the MAR image, which is provided in the step S108.

In FIG. 6, the letter “P” indicates projection, and the letter “R” indicated reconstruction.

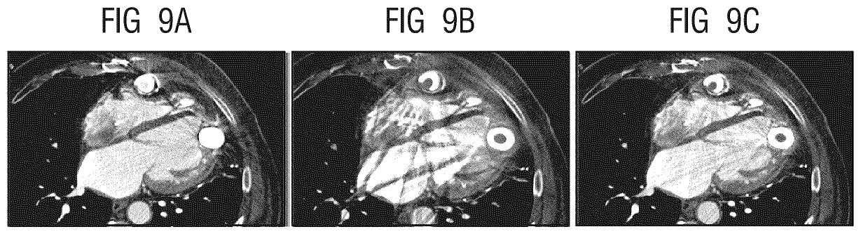

The metal detection algorithm at reference sign S104 in FIG. 6 and the DL MAR algorithm comprising the DL-based prior image calculation at reference sign S106-P may be trained independently and/or jointly. In an exemplary embodiment, a joint network architecture (e.g., comprising the, in particular DL, metal detection algorithm followed by the DL-based prior image calculation and/or DL MAR algorithm) is trained with slice-wise material-specific segmentations of the following tissue and material types: 1) air of the background, 2) air inside the human body (e.g., within the airways and lung), 3) soft tissue (including muscle, fat, and/or inner organs), 4) all bone, 5) metal implants, and (optionally if present) 6) contrast-agent. Masks (also denoted as labels) are in this embodiment manually pre-generated case-wise by identifying and classifying voxel-wise tissue types to generate connected material-regions. Prior anatomical and Hounsfield (and/or HU) information from the slices without metal is used by the generated connected regions to pre-generate the material masks in the metal artifact-affected slices. The final slide-wise segmentation masks are, in this embodiment, coming from manual reader annotations by correcting and finalizing the pre-generated, region-wise segmentation masks. The multi-mask (also: multi-label) neural network training with the defined six (6) labels has the same masks (also: labels) as its output, which are used in distinct steps. Voxels, which belong to the metal class 5) are used to replace, according to the inventive technique, the conventional (e.g., HU) threshold-based metal detection. The conventional (e.g., HU) threshold-based bone segmentation is, according to the inventive technique, replaced by the output mask of the neural network corresponding to label 4) bone and, if present, label 6) contrast-agent. Using conventional methods, contrarily to the inventive technique, contrast agent is not handled separately, which leads to corrupt contrast agent regions after MAR, as exemplified in FIG. 9B.

Some algorithm steps may remain unchanged compared to the prior art. By the inventive technique, in particular the first and third steps described below are novel. The exemplary embodiment of the inventive technique in FIG. 6 may comprise the following steps, performed on all image slices of a given CT input volume: Firstly, the metal mask is determined (e.g., calculated) at reference sign S104 by a first U-Net or similar neural network, classifying each voxel of the slice into metal voxel and/or non-metal voxel. Secondly, metal projections are acquired from forward-projecting the binarized metal mask from the first step. Thirdly, the prior image calculation is performed at reference sign S106-P by a U-Net shaped deep neural network (or a similar neural network), determining (e.g., calculating) segmentation masks of the air, bone, and/or soft-tissue regions. Fourthly, the uncorrected sinogram (acquired from forward-projecting the uncorrected input image received at reference S102) is normalized in the sinogram domain by the forward-projected prior image from the third step and/or from reference sign S106-P. Fifthly, within all projections of the metal trace (from the second step at reference sign S104), linear interpolation is performed for each projection angle between the boundaries of the metal trace, as indicated at reference sign S106-I. Sixly, the interpolated sinogram is denormalized (not shown in FIG. 6) again by the prior sinogram. Seventhly, the final image composition is acquired by an image-based frequency split at reference sign S106-F. The fifth to seventh step may correspond to method steps as described in [5], [9] or [10].

As indicated at reference sign 601, in one variant, in addition to reconstructing S102-2 the (in particular uncorrected) volumetric image from a projection representation (such as a sinogram), the projection representation (e.g., the sinogram) may be directly used in the step S106-I of performing the interpolation. In this case, the uncorrected volumetric image reconstruction need not be transformed back into the projection representation (e.g., the sinogram).

The following benefits can result from the inventive technique (e.g., comprising the method 100, and/or the computing device 200): Less user-input and/or user-interaction are required at the scanner (and/or on the modality). The workflow is highly simplified and sped up at the scanner (also: modality). By removing the metal artifact reduction from the scanner (and/or modality) to a later point in the imaging chain, the operator does not need to specify the necessity for metal artifact reduction anymore and/or does not need to specify the implant setting within the current scanner-deployed iMAR. As a result, fewer reconstruction tasks need to be created, assigned, and modified manually, simplifying the scanner operation. Alternatively or in addition, the inventive technique can ensure that less reconstructions are required at the scanner (and/or on the modality). Adding up to the base image reconstruction at the scanner (also: on the modality), image-based reconstruction tasks are conventionally consuming computational power, storage, and require data transfer capacity. E.g., on the NAEOTOM Alpha, individual clinical sites use the scanner (also: modality) for the computation of reconstructions besides the standard reconstructions, because the high spatial resolution, the small slice thickness, and/or the intrinsic spectral resolution consume most of the computational power. An outsourced computation of a MAR image fully resolves all tasks, which occur because of metal artifact reduction image series. Alternatively or in addition, the inventive technique allows for less user-input and/or user-interaction required on the PACS. Conventionally, during diagnostic image viewing, either the PACS automatically hangs the available image series, based on regular expressions from the series description or DICOM tags, or the user (and/or operator) must align those manually. The manual hanging is usually performed by identifying image series based on a small review thumbnail and parts of the series description and then dragging them manually into the chosen review layout. More image series (as from metal artifact reduction on the original images series or combinations with additional postprocessing, such as VMI) add complexity to this procedure, as series can't be identified uniquely based on the automatic hanging protocols or the manual image identification. This problem is resolved by the inventive technique by embedding the overlay of the precomputed MAR-corrected image to the PACS viewer.

A functionality of the inventive technique is characterized in that, and/or can be detected in other software vendors, as soon as a PACS or another algorithm-hosting or image-interpretation platform outside the scanner (and/or modality) is capable of reducing metal artifacts (and/or other image-based reconstruction tasks), e.g., on conventional CT volume data.

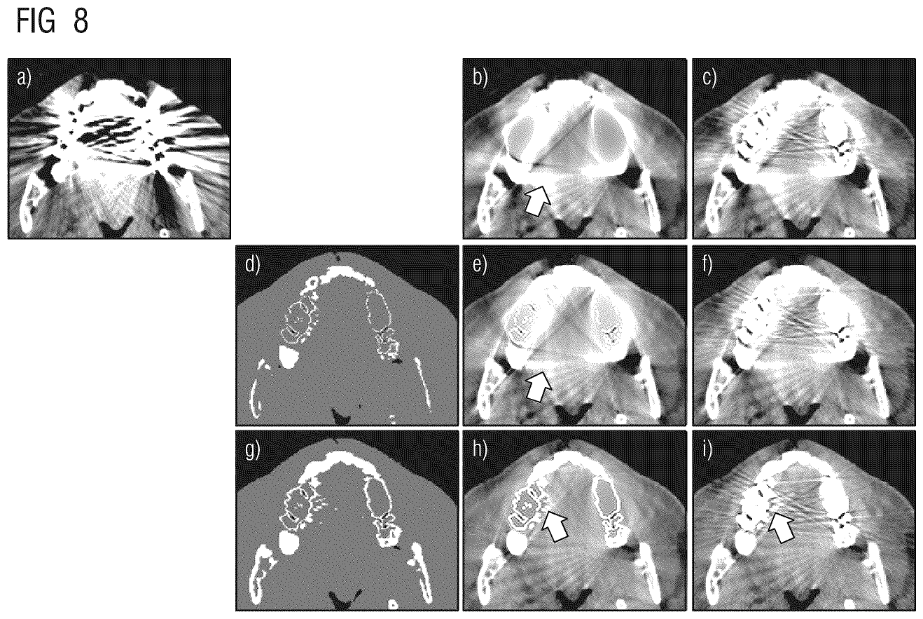

By the inventive technique (e.g., comprising the method 100, and/or the computing device 200), the image quality of the reconstructed image can strongly increase with the quality of the prior image, e.g., as used with the normalized sinogram interpolation of [5]. Image-based thresholding often fails to fully segment bone and air within the prior image, as exemplarily shown within FIGS. 7A, 7B, 7C and 7D. If not enough of the bone is segmented in the prior image, the sinogram after the normalized sinogram interpolation is inconsistent (e.g., as described in [6]). This causes the image reconstruction to introduce artifacts, which are exemplarily shown in FIG. 8b). A DL-based prior image calculation introduces a better prior image, causing less interpolation-related artifacts. In addition, slices with contrast agent in the same slice as the metal implant also benefit from an increased image quality, as the inventive technique (e.g., using one or more neural networks) aims at differentiating bone from contrast agent.

FIGS. 7A, 7B, 7C and 7D exemplarily show a comparison between different window settings and HU thresholds. FIG. 7A shows a soft tissue window. FIG. 7B shows a bone window. FIG. 7C shows no window and/or the result of a bone detection threshold at 200 HU. FIG. 7D shows no window and/or the result of a bone detection threshold at 650 HU.

The bone detection threshold influences (e.g., the shape and/or size of) the bone mask, as exemplarily shown by the comparison of FIG. 7C and FIG. 7D. Alternatively or in addition, segmentation for obtaining a metal mask is performed in addition.

Even if the trabecular bone around the endoprosthesis is superimposed by artifacts, when viewed within a normal soft tissue window (e.g., with Center/Width=40 HU/300 HU) in FIG. 7A, the information is still present in FIG. 7B when viewed with a wider bone window (e.g., with Center/Width=500 HU/2000 HU). While still visible for the human observer, simple HU thresholds fail to segment the total bone in the presence of metal artifacts. A low HU threshold, which includes most of the bone, misclassifies artifacts as bone, as indicated by the arrow in FIG. 7C, which can carry over to the corrected image. In contrast, higher HU thresholds don't include the bone structures in total (and/or not at all), as indicated by the arrow in FIG. 7D.

FIG. 8 illustrates that the image quality after metal artifact reduction strongly depends on the quality of the prior image. FIG. 8a) in the first column shows an uncorrected image. The second column shows different prior images in the rows. The third and fourth columns show the impact of the prior image onto the interpolated image and the frequency split image with the different prior images in the rows. In the first row, no prior image is used, as indicated by the empty space between FIG. 8a), b) and d). In the second row, an imprecise prior image, FIG. 8d), is used for the interpolation and the generation of the NMAR image, FIG. 8e), and FSMAR image, FIG. 8f). A better (e.g., more precise) prior image, FIG. 8g), reduces interpolation related hyperdense artifacts in the NMAR image, FIG. 8h), as indicated by the arrows in FIGS. 8b), 8e), 8h) and/or 8i) in the third column and/or third row. In addition, it can be seen that a threshold-based bone segmentation often introduces wrong areas to the prior image, as indicated in FIG. 8g), which carry over to the image after metal artifact reduction, as illustrated in FIG. 8i). This effect can be reduced by applying a DL-based prior image calculation, increasing the overall image quality.

FIGS. 9A, 9B and 9C illustrate the influence of the segmented prior image to the final image quality after metal artifact reduction. In FIG. 9A, an uncorrected image is shown. FIG. 9B shows a conventional inconsistent segmentation of the contrast agent in the prior image. FIG. 9C shows a more consistent, but still not perfect segmentation of the contrast agent in the prior image.

Independent of the grammatical term usage, individuals with male, female or other gender identities are included within the term.

Wherever not already described explicitly, individual embodiments, or their individual aspects and features, described in relation to the drawings can be combined or exchanged with one another without limiting or widening the scope of the described invention, whenever such a combination or exchange is meaningful and in the sense of this invention. Advantages which are described with respect to a particular embodiment of present invention or with respect to a particular figure are, wherever applicable, also advantages of other embodiments of the present invention.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

### CITED PRIOR ART

[1] Kassenärztliche Bundesvereinigung, 2022. Jahrbuch 2022-statistische Basisdaten zur vertragszahnärztlichen Versorgung.
[2] Statistisches Bundesamt, 2022. Fallpauschalbezogene Krankenhausstatistik (DRG-Statistik). Operationen und Prozeduren der vollstationären Patientinnen und Patienten in Krankenhäusern (4-Steller).
[3] Boas, F. E. & Fleischmann, D., 2012. CT artifacts: causes and reduction techniques. Imaging in medicine, 4(2), pp. 229-240.
[4] Kachelrieß and Krauss, 2021. White paper—Iterative metal artifact reduction (iMAR). Technical principles and clinical results in radiation therapy. Siemens Healthineers.
[5] Meyer, E. et al., 2010. Normalized metal artifact reduction (NMAR) in computed tomography. Medical physics, 37(10), pp. 5482-5493.
[6] Anhaus, J. A. et al., 2022. Iterative metal artifact reduction on a clinical photon counting system—technical possibilities and reconstruction selection for optimal results dependent on the metal scenario. Physics in Medicine & Biology, 67(11), p.115018.
[7] Anhaus, J. A. et al., 2023. A nonlinear scaling-based normalized metal artifact reduction to reduce low-frequency artifacts in energy-integrating and photon-counting CT. Medical Physics, 50(8), pp. 4721-4733.
[8] Ronneberger, O., Fischer, P. & Brox, T., 2015. U-net: Convolutional networks for biomedical image segmentation. In International Conference on Medical image computing and computer-assisted intervention. pp. 234-241.
[9] Meyer, E. et al., 2012. Frequency split metal artifact reduction (FSMAR) in computed tomography. Medical physics, 39(4), pp. 1904-1916.
[10] Anhaus, J. et al., 2022. Nonlinearly scaled prior image-controlled frequency split for high-frequency metal artifact reduction in computed tomography. Medical Physics.

1. A computer-implemented method for providing a metal artifact reduced volumetric image reconstruction, the method comprising:
receiving at least one volumetric image reconstruction of a medical scan from a scanner pertaining to a volumetric medical imaging modality;
detecting, via a metal detection algorithm, a metal comprised within the at least one volumetric image reconstruction;
performing, responsive to the detecting of the metal and via a deep learning metal artifact reduction algorithm, a metal artifact reduction on the received at least one volumetric image reconstruction; and
providing the metal artifact reduced at least one volumetric image reconstruction.

2. The method of claim 1, wherein the receiving the at least one volumetric image reconstruction of the medical scan comprises:
receiving raw data of the medical scan, the raw data being at least one of projection data or primary thin slice volume data; and
reconstructing the raw data to obtain the at least one volumetric medical image reconstruction.

3. The method of claim 1, further comprising:
receiving or extracting an indication of an existence of the metal comprised within the at least one volumetric image reconstruction.

4. The method of claim 1, wherein the metal detection algorithm comprises at least one of,
a deep learning metal detection algorithm, or
at least one of,
a neural network based metal detection;
a neural network based metal segmentation; and
an empirical image analysis of at least one of an image domain, a projection domain, an image frequency domain, or a projection frequency domain in view of an existence of the metal within the at least one volumetric image reconstruction.

5. The method of claim 1, wherein the deep learning metal artifact reduction algorithm comprises at least one of:
a neural network based metal artifact reduction;
a neural network based at least one of tissue segmentation or air segmentation; and
an empirical image analysis of at least one of an image domain, a projection domain, an image's frequency domain, or a projection frequency domain in view of an existence of at least one of tissue or air.

6. The method of claim 1, wherein at least one of the detecting the metal, the performing the metal artifact reduction, or the providing the metal artifact reduced at least one volumetric image reconstruction are performed responsive to at least one of:
the receiving of the at least one volumetric image reconstruction from the scanner;
a worklist of a user including assessing the medical scan; and
a user accessing at least one of the received at least one volumetric image reconstruction, or a case comprising the received of the at least one volumetric image reconstruction.

7. The method of claim 1, wherein at least one of the detecting the metal, the performing the metal artifact reduction, or the providing the metal artifact reduced at least one volumetric image reconstruction are performed at least one of:
within a picture archiving and communicating system (PACS);
at a digital image communications and communications in medicine (DICOM) node;
a computing platform; or
at a cloud server.

8. The method of claim 1, further comprising:
storing the provided metal artifact reduced at least one volumetric image reconstruction.

9. The method of claim 1, further comprising at least one of:
receiving, at a user interface, UI, a viewing request in relation to the at least one volumetric image reconstruction; and
displaying an image of the received at least one volumetric image reconstruction and/or an image of the metal artifact reduced at least one volumetric image reconstruction.

10. The method of claim 1, wherein the volumetric medical imaging modality comprises computed tomography.

11. The method of claim 10, wherein the performing the metal artifact reduction comprises at least one of:
determining at least one prior image, wherein the at least one prior image comprises regions of the at least one volumetric image reconstruction without detected metal;
performing an interpolation between regions of the at least one volumetric image reconstruction that do not comprise the detected metal using a representation of a projection domain; and
superimposing the detected metal onto at least one of the at least one volumetric image reconstruction or onto the interpolated at least one volumetric image reconstruction.

12. The method of claim 1, further comprising at least one of:
providing a text draft for inclusion in a medical report on the medical scan; and
receiving a user input in view of the provided text draft.

13. The method of claim 1, wherein at least one of the metal detection algorithm or the deep learning metal artifact reduction algorithm is trained using annotated training data.

14. The method of claim 13, wherein at least one of,
the metal detection algorithm is trained based on annotated training data comprising manually provided metal masks associated with volumetric image reconstructions of medical scans; or
the deep learning metal artifact reduction algorithm is trained based on annotated training data comprising tissue masks, air masks, and optionally contrast agent masks, associated with volumetric image reconstructions of medical scans, preferably wherein the tissue masks comprise soft tissue masks and bone tissue masks.

15. A computing device configured to provide a metal artifact reduced volumetric image reconstruction, the computing device comprising:
a first interface configured to receive at least one volumetric image reconstruction of a medical scan from a scanner pertaining to a volumetric medical imaging modality;
a metal detection module configured to detect, via a metal detection algorithm, a metal comprised within the at least one volumetric image reconstruction;
a metal artifact reduction module configured to perform, responsive to the detecting of the metal and via a deep learning metal artifact reduction algorithm, a metal artifact reduction on the received at least one volumetric image reconstruction; and
a second interface configured to provide the metal artifact reduced at least one volumetric image reconstruction.

16. The computing device of claim 15, wherein the first interface is configured to
receive raw data of the medical scan, the raw data being at least one of projection data or primary thin slice volume data; and
reconstruct the raw data to obtain the at least one volumetric medical image reconstruction.

17. A system configured to provide a metal artifact reduced volumetric image reconstruction, the system comprising:
at least one scanner pertaining to a volumetric medical imaging modality;
the at least one computing device of claim 15, wherein the first interface is configured to receive the at least one volumetric image reconstruction of the medical scan from the at least one scanner; and
at least one graphical user interface configured to display the received at least one volumetric image reconstruction and the provided metal artifact reduced at least one volumetric image reconstruction.

18. The method of claim 9, wherein the displaying at least one of the image of the received at least one volumetric image reconstruction or the image of the metal artifact reduced at least one volumetric image reconstruction comprises the user selecting an image view of the received at least one volumetric image reconstruction, and a corresponding image view of the metal artifact reduced at least one volumetric image reconstruction is displayed side-by-side; or
overlayed with the image view of the received at least one volumetric image reconstruction.

19. The method of claim 11, wherein
the determining the at least one prior image is based on a multi-mask segmentation, the multi-mask comprising one or more air masks, and one or more tissue masks,
the performing the interpolation is based on projected representations of the at least one volumetric image reconstruction, the at least one prior image and the detected metal, and
the superimposing comprises modifying a low-frequency part of the at least one volumetric image reconstruction by the performed interpolation between regions that do not comprise the detected metal.

20. The method of claim 12, wherein the user input comprises an acceptance, a rejection, or a modification of the text draft.
